(19) United States
(12) Patent Application Publication
(10) Pub. No.: US 2022/0133954 A1
(43) Pub. Date: May 5, 2022

(54) PENIS AUGMENTATION

(71) Applicant: The Duke Limited, Rio de janeiro (BR)

(72) Inventor: Marcos Sforza, Rio de janeiro (BR)

(21) Appl. No.: 17/088,335

(22) Filed: Nov. 3, 2020

(51) Int. Cl. A61L 27/36; A61L 27/38

(52) U.S. Cl. CPC A61L 27/3604; A61L 27/3895; A61L 2430/40; A61L 27/3839; A61L 27/3834

(57) ABSTRACT

The present invention provides a method of penis enlargement. The method comprises administering to a site of penis of a subject a graft enriched with adipose derived regenerative cells.

### FIELD OF THE INVENTION

The present invention relates to a method for penis enlargement and graft thereof.

### BACKGROUND OF THE INVENTION

Organ augmentation generally involve a tissue graft application after which regenerative cells, often times adipose derived regenerative cells (ADRCs), also called as adipose derived stem cells (ADSCs), due to availability, form organ cells along with vascularization in the graft, which invades further into the organ tissue upon further development. Certain volume of adipose tissue in the organ is often required, as adipose tissue is a reservoir of regenerative cells. As such, organ augmentation is often performed on soft tissue organs such as breasts and buttock, for example, as adipose tissue, which is in abundance in these organs, is readily available for vascularization.

Penis augmentation is not only a matter of choice, but often is a matter of necessity as too limited a size, e.g., length or diameter, may negatively impact the egg impregnation process. Penis augmentation poses unique challenges, however. There is no or little, if any, adipose tissue on a penis (see FIG. 1), and fat grafting requires a fat tissue environment to survive. As such, viability of a graft on penis is a key issue that remains to be addressed. For this reason, penis augmentation remains largely unknown.

Therefore, there is a need for methods and grafts for penis enlargement.

The embodiments below address the above-identified issues and needs.

### SUMMARY OF THE INVENTION

In one aspect of the present invention, it is provided a method of penis enlargement, comprising:

administering to a subject in need thereof a graft that is enriched with adipose-derived regenerative cells (“ADRCs”),

wherein administering comprising delivering to a site of penis of the subject under Deep fascia.

In some embodiments of the invention method, optionally in combination with any or all the various embodiments disclosed herein, the graft enriched with ADRCs comprises a volume of stromal vascular fraction (“SVF”) obtained from adipose tissue harvested from the subject.

In some embodiments of the invention method, optionally in combination with any or all the various embodiments disclosed herein, the SVF comprises 1,000 to 1,000,000 cells per mL.

In some embodiments of the invention method, optionally in combination with any or all the various embodiments disclosed herein, the SVF is obtained by a process that does not use enzymatic digestion of the adipose tissue.

In some embodiments of the invention method, optionally in combination with any or all the various embodiments disclosed herein, the graft further comprises a volume of the adipose tissue.

In some embodiments of the invention method, optionally in combination with any or all the various embodiments disclosed herein, the graft comprises from 0.1 mL to 10 mL SVF.

In some embodiments of the invention method, optionally in combination with any or all the various embodiments disclosed herein, the site comprises a plurality of application points provided by a prescription.

In some embodiments of the invention method, optionally in combination with any or all the various embodiments disclosed herein, the SVF is obtained by a SPING device.

In some embodiments of the invention method, optionally in combination with any or all the various embodiments disclosed herein, the subject is a human being.

In some embodiments of the invention method, optionally in combination with any or all the various embodiments disclosed herein, the subject is an animal.

In some embodiments of the invention method, optionally in combination with any or all the various embodiments disclosed herein, the graft is an autologous graft or allogenic graft.

In some embodiments of the invention method, optionally in combination with any or all the various embodiments disclosed herein, the graft is generated in situ, and the SVF is generated in situ.

### BRIEF DESCRIPTION OF THE DRAWINGS

FIG. 1 shows the anatomy of human penis.

FIG. 2 shows the photo pictures of test results of cells separated by a SPING device and cells by a commercially available device.

### DETAILED DESCRIPTION OF THE INVENTION

### Definitions

As used herein, the term “enhanced mechanical separation” refers to an enhanced degree of separation of stromal vascular fraction from adipose tissue without the aid of chemical or biochemical agents such as an enzyme. The use of enzyme for cell separation is a technique to separate cells from adipose tissue called for by a need to achieve such, which itself indicates that without the using an agent, it would be much harder to separate cells from adipose tissue. In this context, the term “less trauma to cells” refers to a lesser degree of trauma to cells relative to the degree of trauma to cells caused by cell separation from adipose tissue using a technology different than the one disclosed in this application, e.g., separation with enzymatic digestion or strong mechanical agitation.

As used herein, the term “condition” refers to a medical or cosmetic condition that can be addressed by ADSCs or SVF or a graft containing any of these.

As used herein, the term “in situ” refers to a method of processing a volume of tissue from a subject and/or using a component of the tissue such as SVF without delay or substantial delay prior to use on the subject or another subject. In this context, without delay or substantial delay shall mean within a single procedure or a sequential of procedures of a single design of operations on a single subject or different subjects.

Penis Anatomy

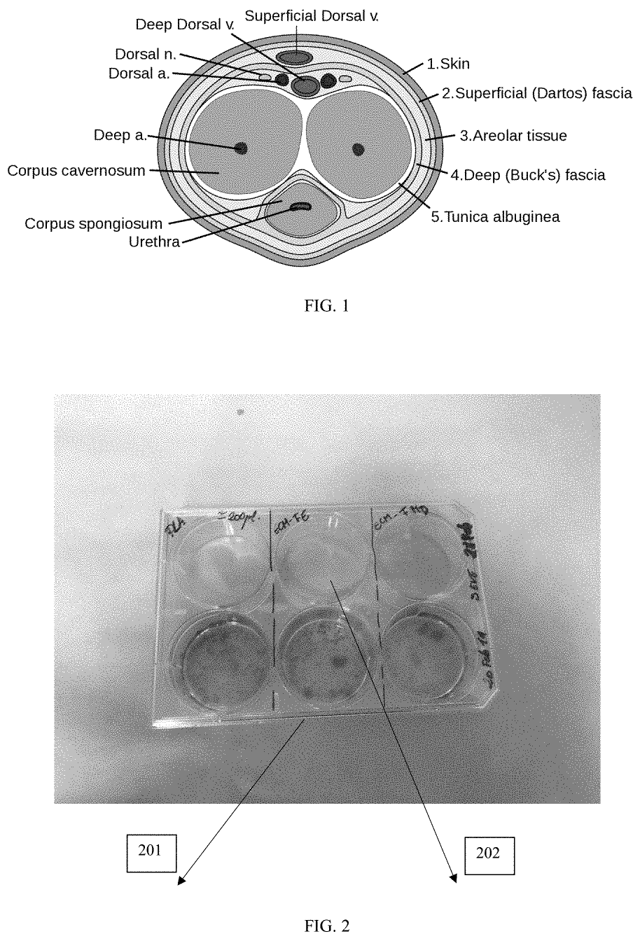

Penis is an organ that lacks adipose tissue. FIG. 1 shows a view of penis anatomy. Anatomically, penis includes:

layers of the following tissues, which are, skin, superficial (Dartos) fascia, areolar tissue, deep (Buck's) fascia, and tunica albuginea;
a superficial dorsal vein, which is between the superficial fascia layer and the deep fascia layer and surrounded by areola tissue;
two dorsal nerves, two dorsal arteries, and a deep dorsal vein, which all are between the deep fascia layer and the tunica albuginea layer;
two corpus cavernosum tissue parts, each with a deep artery therein, one of which is on the left, the other on the right;
corpus spongiosum, which is surrounded by tissue of the deep fascia layer; and
urethra, which is surrounded by the corpus spongiosum.

Penis Enlargement

In one aspect of the present invention, it is provided a method of penis enlargement, comprising:

administering to a subject in need thereof a graft that is enriched with adipose-derived regenerative cells (“ADRCs”), wherein administering comprising delivering to a site of penis of the subject under Deep fascia.

In some embodiments of the invention method, optionally in combination with any or all the various embodiments disclosed herein, the graft enriched with ADRCs comprises a volume of stromal vascular fraction (“SVF”) obtained from adipose tissue harvested from the subject.

In some embodiments of the invention method, optionally in combination with any or all the various embodiments disclosed herein, the SVF comprises 1,000 to 1,000,000 cells per mL.

In some embodiments of the invention method, optionally in combination with any or all the various embodiments disclosed herein, the SVF is obtained by a process that does not use enzymatic digestion of the adipose tissue.

In some embodiments of the invention method, optionally in combination with any or all the various embodiments disclosed herein, the graft further comprises a volume of the adipose tissue.

In some embodiments of the invention method, optionally in combination with any or all the various embodiments disclosed herein, the graft comprises from 0.1 mL to 10 mL SVF.

In some embodiments of the invention method, optionally in combination with any or all the various embodiments disclosed herein, the site comprises a plurality of application points provided by a prescription.

In some embodiments of the invention method, optionally in combination with any or all the various embodiments disclosed herein, the SVF is obtained by a SPING device.

In some embodiments of the invention method, optionally in combination with any or all the various embodiments disclosed herein, the subject is a human being.

In some embodiments of the invention method, optionally in combination with any or all the various embodiments disclosed herein, the subject is an animal.

In some embodiments of the invention method, optionally in combination with any or all the various embodiments disclosed herein, the SVF is obtained by a SPING device from a volume of adipose tissue harvested from a site of the subject such that an autologous graft comprising the SVF is made and used in the method of embodiments herein.

In some embodiments of the invention method, optionally in combination with any or all the various embodiments disclosed herein, the SVF is obtained in situ by a SPING device from a volume of adipose tissue harvested from a site of the subject in situ such that an autologous graft comprising the SVF is made and used in the method of embodiments herein.

SPING Devices

SPING is a device for adipose tissue processing, microfragmentation and facilitation of mechanical separation of adipose derived stem cells (“ADSCs”). Generally, a SPING device includes the following components or compartments, which can include:

an upper housing with an inlet,

a lower housing with an outlet,

a filter stack, and

a spiral flow effectuate;

the upper housing and the lower housing are configured to join to form an enclosure that encloses the filter stack and the spiral flow effectuater, and

the spiral effectuate is configured to receive a flower of filtrate from the filter stack and generate a spiral flow of the adipose tissue to minimize a direct shock of the adipose tissue with walls of the filter stacker to promote enhanced mechanical separation with less trauma to cells of the adipose tissue.

In some embodiments of the invention device, optionally in combination with any or all the various embodiments disclosed herein, the filter stack comprises at least one filter having multiple holes of a size that is the same or different, the size ranging from about 0.4 mm to about 3 mm.

In some embodiments of the invention device, optionally in combination with any or all the various embodiments disclosed herein, the filter stack comprises at least one filter having multiple holes of alternating sizes.

In some embodiments of the invention device, optionally in combination with any or all the various embodiments disclosed herein, the filter stack comprises a first filter, a second filter, and a third filter, where—

the first filter having multiple holes of a size that is the same ranging from about 2.2 mm to about 1.45 mm;

the second filter having multiple holes of alternating sizes where one size that is the same ranging from about 1.8 mm to 0.9 mm and another size that is the same ranging from about 1.35 mm to about 0.6 mm; and the third filter having multi holes of a size that is the same ranging from about 1.35 mm to about 0.45 mm.

In some embodiments of the invention device, optionally in combination with any or all the various embodiments disclosed herein, the upper housing and lower housing are joined by a tongue and groove joint joined by ultrasonic welding.

In some embodiments of the invention device, optionally in combination with any or all the various embodiments disclosed herein, the filter stack comprises more than one filters, and the more than one filters are connected via rods or tubes.

In some embodiments of the invention device, optionally in combination with any or all the various embodiments disclosed herein, the inlet and outlet comprise a Luer lock thread.

In some embodiments of the invention device, optionally in combination with any or all the various embodiments disclosed herein, the at least one of the upper housing, lower housing, the filter stack or the spiral flow effectuater is made from polycarbonate or stainless steel.

In some embodiments of the invention device, optionally in combination with any or all the various embodiments disclosed herein, the SPING device is a disposable device.

Certain embodiments of the SPING device and methods of using and making the same are described in International Patent Application No. PCT/IB2020/060324, filed on Nov. 3, 2020. The teaching of this PCT application, including the SPING device and methods of obtaining SVF from a volume of adipose tissue harvested from a subject is incorporated herein by reference in its entirety.

Graft

The graft useable in the present invention can be autologous graft or allogenic graft. In some embodiments, the graft comprises a volume of the stromal vascular fraction (“SVF”) generated by a method of invention, the method comprising

subjecting a volume of adipose tissue to fragmentation and separation by a device to generate a volume of fragmented adipose tissue; and

subject the fragmented adipose tissue to centrifuging to generate a volume of the SVF,

wherein the device is a device for adipose tissue processing, microfragmentation and facilitation of mechanical separation of adipose derived stem cells (“ADSCs”), the device comprising: an upper housing with an inlet, a lower housing with an outlet, a filter stack, and a spiral flow effectuate, where—
the upper housing and the lower housing are configured to join to form an enclosure that encloses the filter stack and the spiral flow effectuater; and
the spiral effectuate is configured to receive a flower of filtrate from the filter stack and generate a spiral flow of the adipose tissue to minimize a direct shock of the adipose tissue with walls of the filter stacker to promote enhanced mechanical separation with less trauma to cells of the adipose tissue.

In some embodiments of the invention graft, optionally in combination with any or all the various embodiments disclosed herein, the filter stack comprises at least one filter having multiple holes of a size that is the same or different, the size ranging from about 0.4 mm to about 3 mm.

In some embodiments of the invention graft, optionally in combination with any or all the various embodiments disclosed herein, the filter stack comprises at least one filter having multiple holes of alternating sizes.

In some embodiments of the invention graft, optionally in combination with any or all the various embodiments disclosed herein, the filter stack comprises a first filter, a second filter, and a third filter, where—
the first filter having multiple holes of a size that is the same ranging from about 2.2 mm to about 1.45 mm;
the second filter having multiple holes of alternating sizes where one size that is the same ranging from about 1.8 mm to 0.9 mm and another size that is the same ranging from about 1.35 mm to about 0.6 mm; and
the third filter having multi holes of a size that is the same ranging from about 1.35 mm to about 0.45 mm.

In some embodiments of the invention graft, optionally in combination with any or all the various embodiments disclosed herein, the upper housing and lower housing are joined by a tongue and groove joint joined by ultrasonic welding.

In some embodiments of the invention graft, optionally in combination with any or all the various embodiments disclosed herein, the filter stack comprises more than one filters, and the more than one filters are connected via rods or tubes.

In some embodiments of the invention graft, optionally in combination with any or all the various embodiments disclosed herein, the inlet and outlet comprise a Luer lock thread.

In some embodiments of the invention graft, optionally in combination with any or all the various embodiments disclosed herein, the at least one of the upper housing, lower housing, the filter stack or the spiral flow effectuater is made from polycarbonate or stainless steel.

In some embodiments of the invention graft, optionally in combination with any or all the various embodiments disclosed herein, the device is a disposable device.

In some embodiments of the invention graft, optionally in combination with any or all the various embodiments disclosed herein, the graft further comprises a pharmaceutically acceptable carrier.

In some embodiments of the invention graft, optionally in combination with any or all the various embodiments disclosed herein, the graft further comprises a volume of adipose tissue.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The following examples illustrate rather than limit the embodiments of the present invention.

### EXAMPLES

### Example 1. Studies on Cell Separation Using SPING Devices: A Comparison Study

Materials and Methods

SPING devices were used for cell separation and cell viability studies. Cell separation and cell viability studies were also performed on a device by TONNARD Technique (“Tonnard Technique device”). Adipose tissue was harvested from 5 female at the abdomen, which was stored at 4° C. for 24 hrs before use.

SPRING prototype plastic: mechanical dissociation 20 passes (MS 20);

SPRING prototype stainless steel: mechanical dissociation 30 passes (MS 30);

Tonnard Technique device: Luer to Luer, 30 passes.

TABLE 1

| | | Data Collection | | | | |
|---|---|---|---|---|---|---|
| | | | | | SVF cells | |
| | | Resuspension | Total | | per gram | |
| | | vol. | nucleated | | adipose | Mean SVF |
| Sample | Notation | (mL) | cells | Dilution | tissue | cell yield |
| SPING plastic | MS20-1 | 1 | 2.24E+06 | 5 | 1.12E+05 | 1.09E+05 |
| 20-1 | | | | | | |
| SPING plastic | MS20-2 | 1 | 2.12E+06 | 5 | 1.06E+05 | |
| 20-2 | | | | | | |
| SPING Metal | MS30-1 | 1 | 2.69E+06 | 5 | 1.34E+05 | 1.37E+05 |
| 30-1 | | | | | | |
| SPING Metal | MS30-2 | 1 | 2.80E+06 | 5 | 1.40E+05 | |
| 30-2 | | | | | | |
| Tonnard-1 | ML10-1 | 1 | 6.72E+05 | 2 | 6.72E+04 | 6.97E+04 |
| Tonnard-2 | ML10-2 | 1 | 7.22E+05 | 2 | 7.22E+04 | |

Table 1 shows the SPING device (metal: stainless steel) provides the best cell separation result and SVF yield. Compared to Tonnard devices, SPING devices produced SVF cells almost twice as many, which is significant.

Cell culture studies were performed on SVF cells obtained by SPING devices in comparison with that on SVF cells obtained by Tonnard devices. After 14 days culture, SVF cells by the SPING device were shown to be alive, while cells by Tonnard devices were shown to be dead (FIG. 2: SPING SVF cells 201, bottom, shown alive via dye, purple; Tonnard SVF cells 202, top, shown dead via dye, no color).

The results shown in Example 1 are even more convincing and demonstrate that SPING devices generate SVF cells almost twice as efficient as compared with the Tonnard devices. The culture cell viability studies clearly demonstrate that due to reduced impact on cells by SPING device and that the SVF are convincingly far more viable as compared with SVF cells obtained by Tonnard devices, which is very significant in penis enlargement applications.

A graft is then made using the SVF cells from Example 1, which is then administered to a site of penis of a patient according to a pattern prescribed by a clinician. The patent can include one or more injection sites or points to achieve penis enlargement results that the clinician sees as appropriate. The graft can be SVF cells alone or in combination with a volume of adipose tissue. The SVF can comprise 0.1 mL to 10 mL of the graft. In some embodiments, the adipose tissue can be from 0.1 to 10 mL or 10 mL-0.1 mL, which the clinician can prescribe as appropriate.

Those skilled in the art will know, or be able to ascertain, using no more than routine experimentation, many equivalents to the specific embodiments of the invention described herein. These and all other equivalents are intended to be encompassed by the following claims.

I claim:

1. A method of penis enlargement, comprising:
administering to a subject in need thereof a graft that is enriched with adipose-derived regenerative cells (“ADRCs”),
wherein administering comprising delivering to a site of penis of the subject under Deep fascia.

2. The method according to claim 1, wherein the graft enriched with ADRCs comprises a volume of stromal vascular fraction (“SVF”) obtained from adipose tissue harvested from the subject.

3. The method according to claim 2, wherein the SVF comprises 1,000 to 1,000,000 cells per mL.

4. The method according to claim 2, wherein the SVF is obtained by a process that does not use enzymatic digestion of the adipose tissue.

5. The method according to claim 1, wherein the graft further comprises a volume of the adipose tissue.

6. The method according to claim 1, wherein the graft comprises from 0.1 mL to 10 mL SVF.

7. The method according to claim 1, herein the site comprises a plurality of application points provided by a prescription.

8. The method according to claim 4, wherein the SVF is obtained by a SPING device.

9. The method according to claim 1, wherein the subject is a human being.

10. The method according to claim 1, wherein the subject is an animal.

11. The method according to claim 1, wherein the graft is an autologous graft.

12. The method according to claim 1, wherein the graft is generated in situ, and the SVF is generated in situ.
